(19) United States
(12) Patent Application Publication
(10) Pub. No.: US 2024/0360399 A1
(43) Pub. Date: Oct. 31, 2024

(54) Methods To Remove Solutes Without Suspension Cell Loss

(71) Applicant: Terumo BCT, Inc., Lakewood, CO (US)

(72) Inventor: Dennis J. HLAVINKA, Lakewood, CO (US)

(21) Appl. No.: 18/637,782

(22) Filed: Apr. 17, 2024

Related U.S. Application Data
(60) Provisional application No. 63/461,989, filed on Apr. 26, 2023.

(51) Int. Cl. C12M 1/00

(52) U.S. Cl. CPC C12M 29/16; C12M 29/04; C12M 29/06; C12M 29/18

(57) ABSTRACT

A method for removing solutes while retaining suspension cells within a bioreactor includes positioning a suspension including cells within a predetermined region of the bioreactor and causing a flow rate across the suspension in the bioreactor, where the flow rate is selected to cause the solutes to move out of the bioreactor while the cells are maintained in the bioreactor.

### CROSS-REFERENCE TO RELATED APPLICATIONS

This application claims the benefit of U.S. Provisional Application No. 63/461,989 filed on Apr. 26, 2023. The entire disclosure of the above application is incorporated herein by reference.

### FIELD

The present disclosure relates to methods for removing large molecular weight solutes without suspension cell loss during cell expansion processes.

### BACKGROUND

This section provides background information related to the present disclosure which is not necessarily prior art.

Hollow-fiber cell expansion systems, also referred to as cell expansion systems and/or hollow-fiber bioreactors, are cell culturing systems including one or more removable bioreactor cartridges used to expand and differentiate cells, including both adherent and non-adherent cell types. The bioreactor cartridges include hollow-fiber membranes including a plurality of semi-permeable hollow fibers. Spaces within the hollow fibers (i.e., the lumen) define an intracapillary space, while a space outside of the hollow fibers defines an extracapillary space. Depending on the permeability of fiber walls of the hollow fibers, large molecular weight solutes (having, for example, a molecular weight greater than or equal to about 20,000 Daltons), such as from a culture media and/or an expanding cell population, may be unable to readily diffuse across the fiber walls, which can lead to undesirable progressive accumulation of the solutes (including, for example, proteins, cytokines, and/or the waste products) within the intracapillary space (or alternatively, or additionally, in the extracapillary space). The accumulation of solutes can have an unfavorable impact on cell viability, proliferation, and/or phenotype. Accordingly, it would be desirable to develop methods for removing undesirable solutes from the intracapillary space (or alternatively, or additionally, in the extracapillary space) without suspension cell loss.

### SUMMARY

This section provides a general summary of the disclosure and is not a comprehensive disclosure of its full scope or all of its features.

The present disclosure provides a method for removing solutes while retaining suspension cells within a bioreactor.

In at least one example embodiment, the method may include positioning a suspension including cells within a predetermined region of the bioreactor and causing a flow rate across the suspension in the bioreactor, where the flow rate is selected to cause the solutes to move out of the bioreactor while the cells are maintained in the bioreactor.

In at least one example embodiment, the positioning of the suspension may include applying a first flow to first ends of the bioreactor and applying a second flow to second ends of the bioreactor, where the first flow and the second flow together may modify a first axial flow from the first ends and a second axial flow from the second ends such that the suspension is positioned within the predetermined region of the bioreactor.

In at least one example embodiment, the first flow may have a first flow rate and the second flow may have a second flow rate that is different from the first flow rate.

In at least one example embodiment, the second flow rate may be less than the first flow rate.

In at least one example embodiment, the second flow rate may be less than one half of the first flow rate.

In at least one example embodiment, the positioning of the suspension may further include positioning the bioreactor in a horizonal or a near horizonal orientation such that the suspension falls onto or near a wall defining the bioreactor.

In at least one example embodiment, after the positioning of the suspension and before the causing of the flow across the suspension, the method may further include stopping all flow.

In at least one example embodiment, after the stopping of all flows through the bioreactor and before the causing of the flow across the suspension, the method may further include positioning flow valves to enable flows to exit the bioreactor.

In at least one example embodiment, before the causing of the flow across the suspension, the method may further include placing the bioreactor at a 45-degee angle or a near 45-degree angle relative to the horizontal.

In at least one example embodiment, the predetermined region of the bioreactor is a position central between first ends and opposing second ends of the bioreactor.

In at least one example embodiment, the predetermined region is perpendicular to a major axis of the bioreactor.

The present disclosure provides another method for removing solutes while retaining suspension cells within a bioreactor.

In at least one example embodiment, the method may include orientating a bioreactor at an angle relative to a horizontal creating upper and lower ends of the bioreactor, positioning a suspension including cells and solutes in a predetermined position within the bioreactor, initiating a recirculation flow rate, and initiating a preselected net ultrafiltration rate.

In at least one example embodiment, the positioning of the suspension may include applying a first flow to lower ends of the bioreactor and applying a second flow to the upper end of the bioreactor, where the first rate and the second flow together may modify a first axial flow from the first ends and a second axial flow from the second ends such that the suspension is positioned within the predetermined region of the bioreactor.

In at least one example embodiment, the first flow may have a first flow rate and the second flow may have a second flow rate that is different from the first flow rate.

In at least one example embodiment, the second flow rate may be less than the first flow rate.

In at least one example embodiment, the second flow rate may be less than one half of the first flow rate.

In at least one example embodiment, after the positioning of the suspension in the predetermined position within the bioreactor and before the initiating of the recirculation flow rate, the method may further include stopping all flow rates.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1 is an illustration of an example cell expansion system having a bioreactor in accordance with at least one example embodiment;

FIG. 2 is an illustration of an example bioreactor that shows circulation paths through the bioreactor, and which may be incorporated into a cell expansion system, like the cell expansion system illustrated in FIG. 1, in accordance with at least one example embodiment;

FIG. 3 is an illustration of an example rocking device configured to be used with a cell expansion system, like the cell expansion system illustrated in FIG. 1, to move a bioreactor, like the bioreactor of FIG. 2, in accordance with at least one example embodiment;

FIG. 4 is a schematic illustrating example flow paths of an example cell expansion system, like the cell expansion system illustrated in FIG. 1, in accordance with at least one example embodiment;

FIG. 5 is a flowchart illustrating an example method for removing solutes from an intracapillary space (or alternatively, or additionally, in the extracapillary space) of a bioreactor, like the bioreactor illustrated in FIG. 2, in accordance with at least one example embodiment;

FIG. 6 is a simplified illustration of the example method illustrated in FIG. 5 as applied to a single hollow fiber of the bioreactor in accordance with at least one example embodiment;

FIG. 7 is a flowchart illustrating another example method for removing solutes from an intracapillary space (or alternatively, or additionally, in the extracapillary space) of a bioreactor, like the bioreactor illustrated in FIG. 2, in accordance with at least one example embodiment;

FIG. 8 is a simplified illustration of the example method illustrated in FIG. 7 as applied to a single hollow fiber of the bioreactor in accordance with at least one example embodiment;

FIG. 9 is a flowchart illustrating another example method for removing solutes from an intracapillary space (or alternatively, or additionally, in the extracapillary space) of a bioreactor, like the bioreactor illustrated in FIG. 2, in accordance with at least one example embodiment;

FIG. 10 is a simplified illustration of the example method illustrated in FIG. 9 as applied to a single hollow fiber of the bioreactor in accordance with at least one example embodiment;

FIG. 11 is a flowchart illustrating another example method for removing solutes from an intracapillary space (or alternatively, or additionally, in the extracapillary space) of a bioreactor, like the bioreactor illustrated in FIG. 2, in accordance with at least one example embodiment; and

FIGS. 12A and 12B are simplified illustrations of the example method illustrated in FIG. 11 as applied to a single hollow fiber of the bioreactor in accordance with at least one example embodiment.

Corresponding reference numerals indicate corresponding parts throughout the several views of the drawings.

### DETAILED DESCRIPTION

Example embodiments will now be described more fully with reference to the accompanying drawings.

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected, or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers, and/or sections, these elements, components, regions, layers, and/or sections should not be limited by these terms. These terms may be only used to distinguish one element, component, region, layer or section from another region, layer, or section. Terms such as “first,” “second,” and other numerical terms when used herein do not imply a sequence or order unless clearly indicated by the context. Thus, a first element, component, region, layer, or section discussed below could be termed a second element, component, region, layer, or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

Various components are referred to herein as “operably associated.” As used herein, “operably associated” refers to components that are linked together in operable fashion and encompasses embodiments in which components are linked directly, as well as embodiments in which additional components are placed between the linked components. “Operably associated” components can be “fluidly associated.” “Fluidly associated” refers to components that are linked together such that fluid can be transported between them. “Fluidly associated” encompasses embodiments in which additional components are disposed between the two fluidly associated components, as well as components that are directly connected. Fluidly associated components can include components that do not contact fluid but contact other components to manipulate the system (e.g., a peristaltic pump that pumps fluids through flexible tubing by compressing the exterior of the tube).

In this application, including the definitions below, the term “module” or the term “controller” may be replaced with the term “circuit.” The term “module” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

The term code, as used above, may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The term memory circuit is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory, tangible computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general-purpose computer to execute one or more particular functions embodied in computer programs. The functional blocks, flowchart components, and other elements described above serve as software specifications, which can be translated into the computer programs by the routine work of a skilled technician or programmer.

The computer programs include processor-executable instructions that are stored on at least one non-transitory, tangible computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation) (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Swift, Haskell, Go, SQL, R, Lisp, Java®, Fortran, Perl, Pascal, Curl, OCaml, Javascript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

Example embodiments will now be described more fully with reference to the accompanying drawings.

Cell expansions systems are cell culturing systems used to expand and differentiate cells, including both adherent and non-adherent cell types. The present disclosure relates to cell expansion systems and processes, including, for example, cell expansion systems and process like those described in U.S. application Ser. No. 15/943,536, titled EXPANDING CELLS IN A BIOREACTOR, filed Apr. 2, 2018, and published Oct. 2, 2018 and/or U.S. Pat. No. 10,577,585 titled CELL EXPANSION and issued on Mar. 3, 2020, the entire disclosures of which are hereby incorporated by reference.

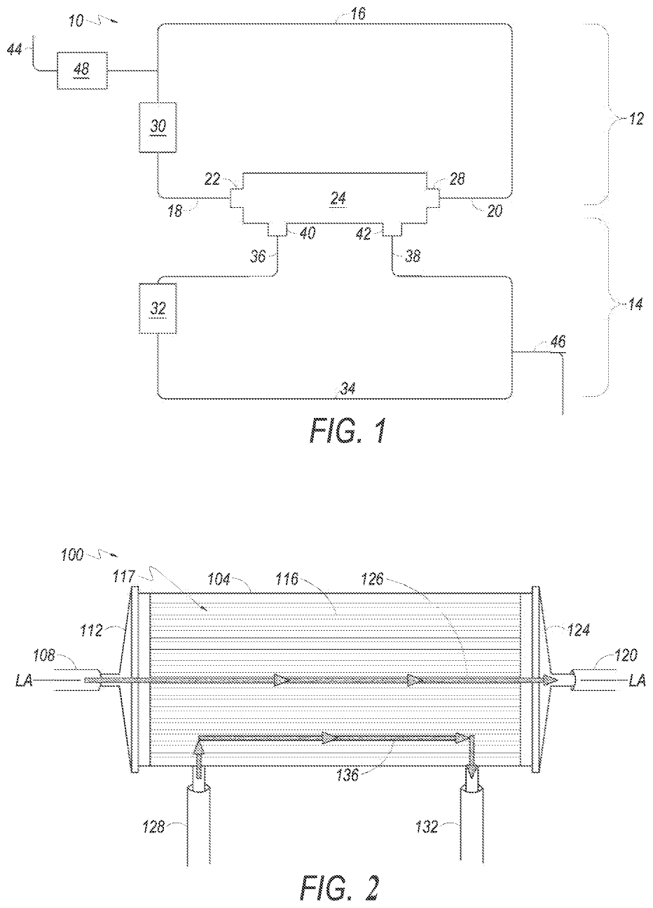

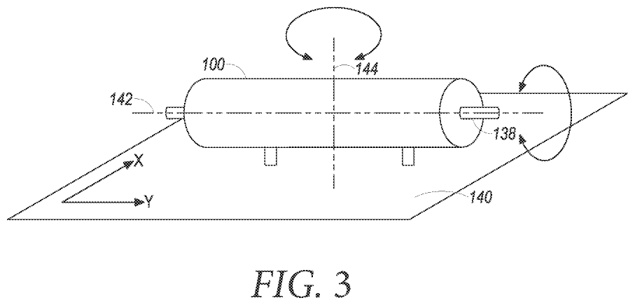

FIG. 1 is an illustration of an example cell expansion system 10. The cell expansion system 10 includes a first fluid circulation path 12 and a second fluid circulation path 14. The first fluid circulation path 12 includes, for example, a first fluid flow path 16 having opposing ends 18 and 20. The first fluid flow path 16 may be in fluid communication with a cell growth chamber 24. For example, the first opposing end 18 of the first fluid flow path 16 may be in fluid communication with a first inlet 22 of the cell growth chamber 24, and the second opposing end 20 may be in fluid communication with first outlet 28 of the cell growth chamber 24. The cell growth chamber 24 may include, or be configured to be receive, a bioreactor (which may also be referred to as a hollow fiber membrane (HFM)) 117 (see FIG. 2). For example, fluid in the first circulation path 12 may flow through an interior of a plurality of hollow fibers 116 of the bioreactor 117. In at least one example embodiment, a first fluid flow control device 30 may be operably coupled to the first fluid flow path 16 and may control the flow of fluid in first fluid circulation path 12.

The second fluid circulation path 14 includes, for example, a second fluid flow path 34 and a second fluid flow control device 32. Like the first fluid flow path 16, the second fluid flow path 34 may have opposing ends 36 and 38. The opposing ends 36 and 38 of second fluid flow path 34 may in fluid communication with an inlet port 40 and an outlet port 42 of the cell growth chamber 24. For example, a first opposing end 36 of the second fluid flow path 34 may be in fluid communication with the inlet port 40 of the cell growth chamber 24, and the second opposing end 38 of the second fluid flow path 34 may be in fluid communication with the outlet port 42. Fluid in the second circulation path 14 may be in contact with an outside of the bioreactor 117 disposed in the cell growth chamber 24. In at least one example embodiment, a second fluid flow control device 32 may be operably coupled to the second fluid flow path 34 and may control the flow of fluid in the second fluid circulation path 14.

The first and second fluid circulation paths 12, 14 may be maintained in the cell growth chamber 24 by way of the bioreactor 117, where fluid in first fluid circulation path 12 flows through an intracapillary (IC) space of the bioreactor 117 and fluid in the second circulation path 14 flows through the extracapillary (EC) space of the cell growth chamber 24. The first circulation path 12 may be referred to as the “intracapillary loop” or “IC loop”. The second fluid circulation path 14 may be referred to as the “extracapillary loop” or “EC loop”. Fluid in first fluid circulation path 12 may flow in either a co-current or counter-current direction with respect to a fluid flow in second fluid circulation path 14.

In at least one example embodiment, a fluid inlet path 44 may be fluidly associated with the first fluid circulation path 12, and a fluid outlet path 46 may be fluidly associated with the second fluid circulation path 14. The fluid inlet path 44 may permit fluid into first fluid circulation path 12, while the fluid outlet path 46 may permit fluid to exit the cell expansion system 10. In at least one example embodiment, as illustrated, a third fluid flow control device 48 may be operably associated with the fluid inlet path 44. Although not illustrated, it should be recognized that, in various other example embodiments, a fourth fluid flow control device may alternatively or additionally be associated operably associated with the first outlet path 46. In at least one example embodiment, the fluid flow control devices (including the first fluid flow control device 30 and/or the second fluid flow control device 32 and/or the third fluid flow control device 48 and/or the fourth fluid flow control device) may include a pump, valve, clamp, or any combination thereof. For example, multiple pumps, valves, and clamps can be arranged in any combination. In at least one example embodiment, the fluid flow control device may be, or include, a peristaltic pump. Fluid circulation paths (including the first fluid circulation path 12 and/or the second fluid circulation path 14) and/or inlet ports (including the fluid inlet port 44) and/or the outlet port (including the fluid outlet port 46) may include any known tubing material, and any kind of fluid-including, for example, buffers, protein containing fluid, and cell-containing fluid-can flow through the various circulation paths (including the first fluid circulation path 12 and/or the second fluid circulation path 14) and/or the inlet paths (including the fluid inlet port 44), and outlet paths (including the fluid outlet port 46). It should be recognized that the terms “fluid,” “media,” and “fluid media” are used interchangeably.

An example cell growth chamber 100 is illustrated in FIG. 2. The cell growth chamber 100 may be used as the cell growth chamber 24 of the cell expansion system 10 illustrated in FIG. 1. The cell growth chamber 100 may have a longitudinal axis (represented by the line LA-LA) and may include a cell growth chamber housing 104. The cell growth chamber housing 104 may have four openings or ports, including, for example, an intracapillary inlet port 108, an intracapillary outlet port 120, an extracapillary inlet port 128, and an extracapillary outlet port 132. A first fluid (which can also be referred to as an intracapillary fluid or media) in a first circulation path (like the first fluid circulation path 12) can enter the cell growth chamber 100 through the intracapillary inlet port 108 at a first fluid manifold end 112 of the cell growth chamber 100 and into and through the intracapillary spaces of a plurality of hollow fibers 116 and out of cell growth chamber 100 through intracapillary outlet port 120, which is located at a second fluid manifold end 124 of the cell growth chamber 100. The fluid path between the intracapillary inlet port 108 and the intracapillary outlet port 120 may define an intracapillary portion 126 of the cell growth chamber 100. A second fluid (which can also be referred to as an extracapillary media or fluid) in a second circulation path (like the second fluid circulation path 14) can enter the cell growth chamber 100 through the extracapillary inlet port 128. This second fluid contacts the extracapillary space or outside of the bioreactor 117 and exits the cell growth chamber 100 via the extracapillary outlet port 132. The fluid path between the extracapillary inlet port 128 and the extracapillary outlet port 132 may define an extracapillary portion 136 of the cell growth chamber 100.

As the second fluid comes into contact with the outside of the hollow fibers 116 small molecules (e.g., ions, water, oxygen, lactate, etc.) may diffuse through the hollow fibers 116 from the interior or intracapillary space of the hollow fibers 116 to the exterior or extracapillary space, or alternatively, or additionally, from the extracapillary space to the intracapillary space. Large molecular weight molecules (e.g., growth factors and/or proteins) are often too large to pass through the membrane walls of the hollow fibers 116 and remain in the intracapillary space (or alternatively, or additionally, in the extracapillary space) of the hollow fibers 116. The mediums defining the first and second fluids may be replaced as needed and may alternatively, or additionally, be circulated through an oxygenator and/or gas transfer module to exchange gasses, as needed. As discussed below, cells for expansion may be contained within the first fluid circulation path 12 and/or the second fluid circulation path 14 and may enter the cell growth chamber 100 on one or both of the intracapillary space or the extracapillary space.

In at least one example embodiment, cells may be seeded (for example, for expansion and/or differentiation and/or harvesting of cord blood derived CD34+ hematopoietic stem/progenitor cells, monocytes, macrophages, hepatocytes, and/or endothelial cells) in the intracapillary space of the hollow fibers 116, while a cell culture medium may be pumped through the extracapillary space of the hollow fibers 116 to deliver nutrients to the cells via hollow fiber membrane perfusion during expansion. However, in at least one other example embodiment, cells for expansion and/or differentiation and/or harvesting may be seeded in the extracapillary space, while the cell culture medium may be pumped through the intracapillary space to deliver nutrients to the cells via hollow fiber membrane perfusion during expansion. In at least one other example embodiment, cells for expansion and/or differentiation and/or harvesting may be seeded in the intracapillary space, while the cell culture medium may be pumped through both the extracapillary space and the intracapillary space. Movement of the cell culture medium through the intracapillary space and/or the extracapillary space can help to remove excess cells, for example, those not adhered to surfaces of the hollow-fiber membrane. In at least one example embodiment, the material used to form the bioreactor 117 may be any biocompatible polymeric material that is capable of being made into the hollow fibers 121. For example, synthetic polysulfone-based materials (e.g., polyethersulfones (PES)) are often used to form the hollow fibers.

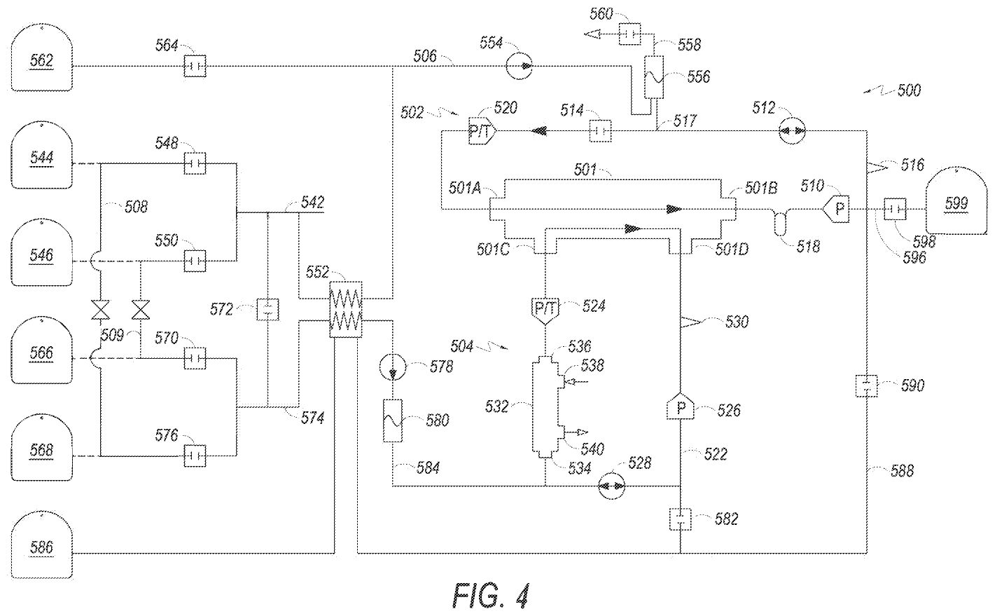

In at least one example embodiment, the cell expansion system 10 may also include a device that is configured to move or “rock” the cell growth chamber 100 relative to other components of the cell expansion system 10. The device may be a rotational and/or lateral rocking device. For example, as illustrated in FIG. 3, the cell growth chamber 100 may be rotationally connected to one or more rotational rocking components 138 and to a lateral rocking component 140. A first rotational rocking component 138 may be rotationally associated with the cell growth chamber 100. For example, the first rotational rocking component 138 may be configured to rotate the cell growth chamber 100 around a first or central rotational axis 142. In at least one example embodiment, the cell growth chamber 100 may be rotated in alternating fashion, including, for example, in a first clockwise direction and then in a second counterclockwise direction around the central axis 142.

Although not illustrated, it should be recognized that in at least one example embodiment, a second rotational rocking component may be configured to move the cell growth chamber 100 about a second rotational axis 144 that passes through a center point of the cell growth chamber 100 normal to the central axis 142. In at least one example embodiment, the cell growth chamber 100 may be rotated in alternating fashion, including, for example, in a first clockwise direction and then in a second counterclockwise direction around the second axis 144. In at least one example embodiment, the cell growth chamber 100 may also be rotated around the second axis 144 and positioned in a horizontal or vertical orientation relative to gravity. The lateral rocking component 140 may be laterally associated with the cell growth chamber 100. For example, a plane of the lateral rocking component 140 may move laterally in the x-direction and y-direction.

The rotational and/or lateral movement of the cell growth chamber 100 may reduce the settling of cells and the likelihood of cells becoming trapped within a portion of the bioreactor 117 disposed in the cell growth chamber 100. In at least one example embodiment, the rate of cells settling in the cell growth chamber 100 may be proportional to the density difference between the cells and the suspension media, according to Stoke's Law. In at least one example embodiment, a 180-degree rotation (fast) with a pause (having, for example, a total combined time of 30 seconds) repeated as described above may help to keep non-adherent cells (for example, t-cells) suspended. A minimum rotation of about 180-degrees may be preferred, however various degrees of rotation, including up to or greater than 360-degrees, may be used. Different rocking components may be used separately or may be combined in any combination. For example, a rocking component that rotates cell growth chamber 100 around central axis 142 may be combined with the rocking component that rotates cell growth chamber 100 around axis 144. Likewise, clockwise and counterclockwise rotation around different axes may be performed independently in any combination.

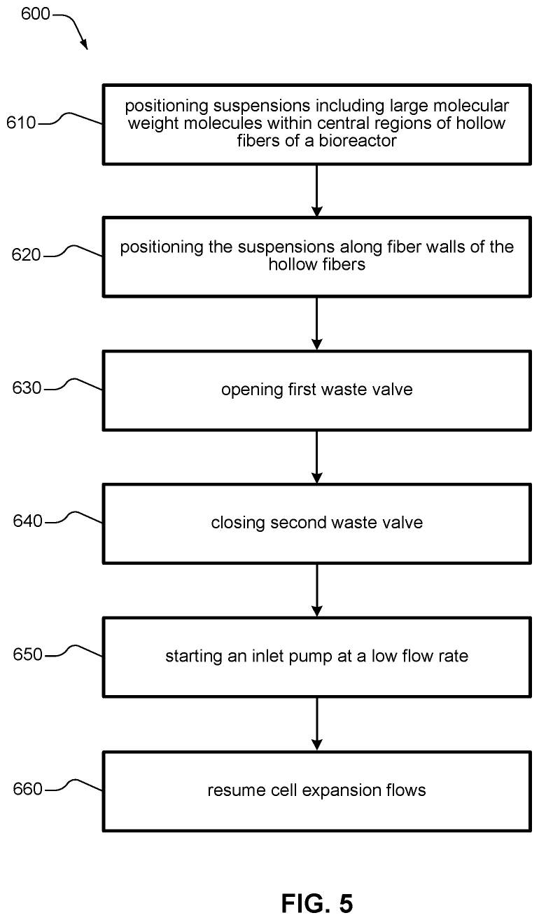

FIG. 4 is a schematic of an example cell expansion system 500, which may be like the cell expansion system 100 illustrated in FIG. 1, that illustrates example flow paths. In at least one example embodiment, the cells may be positioned in the intracapillary space, while a cell culture medium may be pumped through the extracapillary space to deliver nutrients to the cells via hollow fiber membrane perfusion during expansion. It should be recognized, however, in at least one other example embodiment, cells can be positioned in the extracapillary space, while the cell culture medium may be pumped through the intracapillary space to deliver nutrients to the cells via hollow fiber membrane perfusion during expansion. In at least one other example embodiment, cells may be positioned in the intracapillary space, while the cell culture medium may be pumped through both the extracapillary space and the intracapillary space.

As illustrated, the cell expansion system 500 may include a first fluid circulation path 502 (also referred to as the “intracapillary loop” or “IC loop”) and a second fluid circulation path 504 (also referred to as the “extracapillary loop” or “EC loop”). The first fluid flow path 506 may be fluidly associated with a cell growth chamber 501 to form first fluid circulation path 502. The cell growth chamber 501 may be used as the cell growth chamber 24 illustrated in FIG. 1 and/or the cell growth chamber 100 illustrated in FIG. 2. A first fluid may flow into cell growth chamber 501 through an intracapillary inlet port 501A. The first fluid may exit the cell growth chamber via an intracapillary outlet port 501B. In at least one example embodiment, the first fluid circulation path 502 may include a pressure gauge 510 configured to measure a pressure of the first fluid leaving the cell growth chamber 501. In at least one example embodiment, the first fluid circulation path 502 may include an intracapillary circulation pump 512 configured to control a first fluid flow rate. For example, the intracapillary circulation pump 512 may be configured to pump the first fluid in a first direction or a second direction that is opposite to the first direction. In the later instance, the intracapillary outlet port 501B may be used as an inlet, and the intracapillary inlet port 501A as an outlet. In at least one example embodiment, the first fluid circulation path 502 may include a sample port 516 and/or sample coil 518 configured for first fluid sample extraction. In at least one example embodiment, the first fluid circulation path 502 may include a pressure/temperature gauge 520 configured to detect the pressure and/or temperature of the first fluid during operation. In at least one example embodiment, the first fluid may enter the intracapillary loop 502 via valve 514. In at least one example embodiment, a portion of the cells may be flushed from the intracapillary loop 502 into a harvest bag 599, for example, via valve 598. It should be recognized that, in at least one other example embodiment, the first fluid circulation path 502 may include additional or fewer valves, pressure gauges, pressure sensors, temperature sensors, ports, and/or other devices disposed to isolate and/or measure characteristics of the first fluid along portions of the intracapillary loop 502.

A second fluid may flow into cell growth chamber 501 through an extracapillary inlet port 501C. The second fluid may leave the cell growth chamber 501 via an extracapillary outlet port 501D. In at least one example embodiment, the second fluid in the extracapillary loop 504 may contact an exterior facing surface of hollow fibers disposed in the cell growth chamber 501 thereby allowing diffusion of small molecules into and out of the hollow fibers. In at least one example embodiment, the extracapillary loop 504 may include a pressure/temperature gauge 524 configured to measure a pressure and/or temperature of the second fluid before the second fluid enters the cell growth chamber 501. In at least one example embodiment, the extracapillary loop 504 may include a pressure gauge 526 that is configured to measure a pressure of the second fluid, for example, as it leaves the cell growth chamber 501. In at least one example embodiment, the extracapillary loop 504 may include a sample port 530 configured for second fluid sample extraction.

In at least one example embodiment, the extracapillary loop 504 may include an extracapillary circulation pump 528 and an oxygenator or gas transfer module 532. For example, after leaving the cell growth chamber 501, the second fluid may pass through the extracapillary circulation pump 528 and to and through the oxygenator or gas transfer module 532. In at least one example embodiment, the extracapillary circulation pump 528 may be configured to control a second fluid flow rate. For example, like the intracapillary circulation pump 512, the extracapillary circulation pump 528 may be configured to pump the second fluid in a first direction or a second direction that is opposite to the first direction. In the later instance, the extracapillary outlet port 501D may be used as inlet, and the extracapillary inlet port 501C as an outlet.

In at least one example embodiment, the second fluid flow path 522 may be fluidly associated with the oxygenator or gas transfer module 532 via an oxygenator inlet port 534 and an oxygenator outlet port 536. For example, the second fluid may flow into the oxygenator or gas transfer module 532 via the oxygenator inlet port 534 and may leave or exit the oxygenator or gas transfer module 532 via the oxygenator outlet port 536. In at least one example embodiment, the oxygenator or gas transfer module 532 may be configured to add oxygen to and/or remove bubbles from the second fluid. For example, air and/or gas may flow into the oxygenator or gas transfer module 532 via a first filter 538 and may leave or exit (i.e., flow out of) the oxygenator or gas transfer device 532 through a second filter 540. The first and second filters 538, 540 may be configured to reduce or prevent contaminants from entering the oxygenator or gas transfer module 532. The second fluid in the second fluid circulation path 504 may be in equilibrium with gas entering the oxygenator or gas transfer module 532. In at least one example embodiment, air and/or gas may be purged from the cell expansion system 500, for example, during a priming sequence, air and/or gas may be vented to the atmosphere via the oxygenator or gas transfer module 532. It should be recognized that, in at least one other example embodiment, a second fluid circulation path 504 may include additional or fewer valves, pressure gauges, pressure sensors, temperature sensors, ports, and/or other devices disposed to isolate and/or measure characteristics of the second fluid along portions of the extracapillary loop 504.

In at least one example embodiment, an air removal chamber (ARC) 556 may be fluidly associated with the first circulation path 502. The air removal chamber 556 may include one or more ultrasonic sensors. For example, the air removal chamber 556 may include upper sensor and/or lower sensor which are configured to detect air and/or a lack of fluid and/or gas-fluid interface at certain measuring positions within the air removal chamber 556. The upper sensor may be disposed near a first end (e.g., top) of the air removal chamber 556. The lower sensor may be disposed near a second end (e.g., bottom) of the air removal chamber 556. Although ultrasonic sensors are discussed, it should be appreciated that the air removal chamber 556 may include, additionally, or alternatively, one or more other sensors, including, for example, optical sensors. Air and/or gas purged from the cell expansion system 500 during portions of a priming sequence and/or other protocols may vent to the atmosphere out air valve 560 via line 558 that may be fluidly associated with air removal chamber 556.

In at least one example embodiment, the first fluid may include cells (for example, from a first fluid container (which can also be referred to as a first media bag or a first bag) 562 and also fluid media (e.g., intracapillary media or fluid) from a second fluid container (which can also be referred to as a second media bag or a second bag) 546. Materials (i.e., cells and/or intracapillary media) form the first and second fluid containers 562, 546 may enter the first fluid circulation path 502 via a first fluid flow path 506. The first fluid container 562 may be fluidly associated with the first fluid flow path 506 and the first fluid circulation path 502 via valve 564. In at least one example embodiment, the second fluid container 546 and a third fluid container (which can also be referred to as a third media bag or third bag) 544 may be fluidly associated with the first fluid inlet path 542, for example, via valves 548 and 550, respectively, or with a second fluid inlet path 574, for example, via valves 570 and 576, respectively. In at least one example embodiment, the materials from the second fluid container 546 and/or the third fluid container 544 may be in fluid communication with a first sterile sealable input priming path 508 and/or a second sterile sealable input priming path 509.

In at least one example embodiment, a fourth fluid container (which can also be referred to as a fourth media bag or a fourth bag) 568 may include an extracapillary media, and a fifth fluid container (which can also be referred to a fifth media bag or a fifth bag) 566 may include a wash solution. Materials (i.e., extracapillary media and/or wash solution) from the fourth and fifth fluid containers 568, 566 may enter the first fluid circulation path 502 and/or the second fluid circulation path 504. For example, in at least one example embodiment, the fifth fluid container 566 may be fluidly associated with valve 570, where valve 570 is fluidly associated with first fluid circulation path 502, for example, via a distribution valve 572 and a first fluid inlet path 542. In at least one example embodiment, the fifth fluid container 566 may be fluidly associated with the second fluid circulation path 504 via the second fluid inlet path 574 and an extracapillary inlet path 584, for example, by opening valve 570 and closing distribution valve 572. The fourth fluid container 568 may be fluidly associated with valve 576, where valve 576 is fluidly associated with first fluid circulation path 502, for example, via the first fluid inlet path 542 and the distribution valve 572. In at least one example embodiment, the fourth fluid container 568 may be fluidly associated with the second fluid inlet path 574 by opening valve 576 and closing the distribution valve 572. In at least one example embodiment, the first fluid inlet path 542 and/or the second fluid inlet path 574 may be fluidly associated with an optional heat exchanger 552.

In at least one example embodiment, fluid may be advanced to the intracapillary loop 502 from the first fluid inlet path 542 and/or the second fluid inlet path 574 via an intracapillary inlet pump 554, and fluid may be advanced to the extracapillary loop 504 via an extracapillary inlet pump 578. In at least one example embodiment, an air detector 580 may also be associated with the extracapillary inlet path 584. The air detector 580 may include, for example, an ultrasonic sensor. In at least one example embodiment, the first and second fluid circulation paths 502, 504 may be fluidly associated with a waste line 588. For example, when valve 590 is in an open state or position, the intracapillary media may flow through the waste line 588 to a waste bag (also referred to as an outlet bag) 586. When valve 582 is opened, extracapillary media may flow through the waste line 588 to the waste bag 586. In at least one example embodiment, cells may be harvested, for example, via a cell harvest path 596. For example, cells from the cell growth chamber 501 may be harvested by pumping the intracapillary media containing the cells through the cell harvest path 596 and also valve 598 to a cell harvest bag 599.

In at least one example embodiment, as illustrated, the fluid in the first fluid circulation path 502 and second fluid circulation path 504 flows through cell growth chamber 501 in the same direction (i.e., a co-current configuration). Although not illustrated, it should be recognized that, in various other example embodiments, the cell expansion system 500 may also be configured to flow in a counter-current conformation. As illustrated in FIG. 4, fluid in the first fluid circulation path 502 may enter the cell growth chamber 501 at the intracapillary inlet port 501A and may leave or exit the cell growth chamber 501 at the intracapillary outlet port 501B. In at least one example embodiment, the first fluid flow path 506 may be fluidly connected to the first fluid circulation path 502, for example, via connection 517. Connection 517 may be a point or location from which the fluid may flow in opposite directions, for example, based on the direction and flow rates of the intracapillary inlet pump 554 and fluid circulation pump 512. Connection 517 may include any type of fitting, coupling, fusion, pathway, and/or tubing that allows the first fluid flow path 506 to be fluidly associated with the first fluid circulation path 502. In at least one example embodiment, connection 517 may include a T-fitting or coupling and/or a Y-fitting or coupling.

In at least one example embodiment, one or more of the gauges (e.g., pressure gauge 510 and/or pressure/temperature gauge 520 and/or pressure/temperature gauge 524 and/or pressure gauge 526), one or more of the valves (e.g., valve 514 and/or valves 548 and/or valves 550 and/or valve 560 and/or valve 564 and/or valve 570 and/or valve 572 and/or valve 576 and/or valve 582 and/or valve 590 and/or valve 596 and/or valve 598), one or more of the ports (e.g., intracapillary inlet port 501A and/or intracapillary outlet port 501B and/or extracapillary inlet port 501C and/or extracapillary outlet port 501D and/or sample port 516 and/or sample port 530 and/or oxygenator inlet port 534 and/or an oxygenator outlet port 536), one or more of the pumps (e.g., intracapillary circulation pump 512 and/or extracapillary circulation pump 528 and/or intracapillary inlet pump 554 and/or extracapillary inlet pump 578), one or more of the filters (e.g., first filter 538 and/or second filter 540), one or more coils (e.g., sample coil 518), one or more modules (e.g., oxygenator or gas transfer module 532), and/or one or more other components of the cell expansion system 500 may be in electrical communication with a control system (not shown). The control system may include a plurality of nodes, which can include various hardware, firmware, and/or software configured to control and/or communicate with the mechanical, electromechanical, and electrical components of the cell expansion system 500, including for example, a controller and a memory.

The controller (which can also be referred to as a processor), can be of any type of microcontroller, microprocessor, Field Programmable Gate Array (FPGA), Application Specific Integrated Circuit (ASIC), etc. An example controller may be the NK10DN512VOK10 microcontroller, made and sold by N9P USA, Incorporated, which is a microcontroller unit with a 32-bit architecture. Other examples controllers may include, for example, at least one of Qualcomm® Snapdragon® 800 and 801, Qualcomm® Snapdragon® 610 and 615 with 4G LTE Integration and 64-bit computing, Apple® A7 processor with 64-bit architecture, Apple® M7 motion coprocessors, Samsung® Exynos® series, the Intel® Core™ family of processors, the Intel® Xeon® family of processors, the Intel® Atom™ family of processors, the Intel Itanium® family of processors, Intel® Core® i5-4670K and i-4770K 22 nm Haswell, Intel® Core® i5-3570K 22 nm Ivy Bridge, the AMD® FX™ family of processors, AMD® FX-4300, FX-6300, and FX-8350 32 nm Vishera, AMD® Kaveri processors, ARM® Cortex™-M processors, ARM® Cortex-A and ARM926EJ-S™ processors, other industry-equivalent processors, and may perform computational functions using any known or future-developed standard, instruction set, libraries, and/or architecture. The memory can be any type of memory including random access memory (RAM), read only memory (ROM), electrically erasable programmable ROM (EEPROM), a portable compact disc read-only memory (CD-ROM), an optical storage device, a magnetic storage device, any suitable combination of the foregoing, or other type of storage or memory device that stores and provides instructions to program and control the controller.

In at least one example embodiment, protocols for expansion of suspension cells (like T-cells) may implement counter flow (for example, using the intracapillary circulation pump 512 and/or the intracapillary inlet pump 554 as illustrated in FIG. 4) into an intracapillary space (or alternatively, or additionally, counter flow into the extracapillary space may be implemented using the extracapillary circulation pump 528 and/or extracapillary inlet pump 578) of a bioreactor (for example, as included within or defining the first circulation path 12 of the cell growth chamber 24 as illustrated in FIG. 1 and/or the intracapillary portion 126 of the cell growth chamber 100 as illustrated in FIG. 2 and/or the first fluid circulation path 502 of the cell growth chamber 501 as illustrated in FIG. 4) to confine cells within the hollow fibers of the bioreactor (for example like the plurality of hollow fibers 116 disposed within or defining the cell growth chambers 24, 100 as illustrated in FIGS. 1 and 2). Large molecular weight molecules are often too large to pass through the membrane walls of the hollow fibers and remain in the intracapillary space (or alternatively, or additionally, in the extracapillary space) of the hollow fibers. The confinement of the large molecular weight molecules (which are herein referred to as “solutes”) can often lead or progressive accumulation of various proteins, cytokines, and other wase products within the intracapillary space (or alternatively, or additionally, in the extracapillary space) of the hollow fibers, which can unfavorably impact cell viability, proliferation, and phenotype. The solutes can come from, for example, the culture media and/or the expanding cell population.

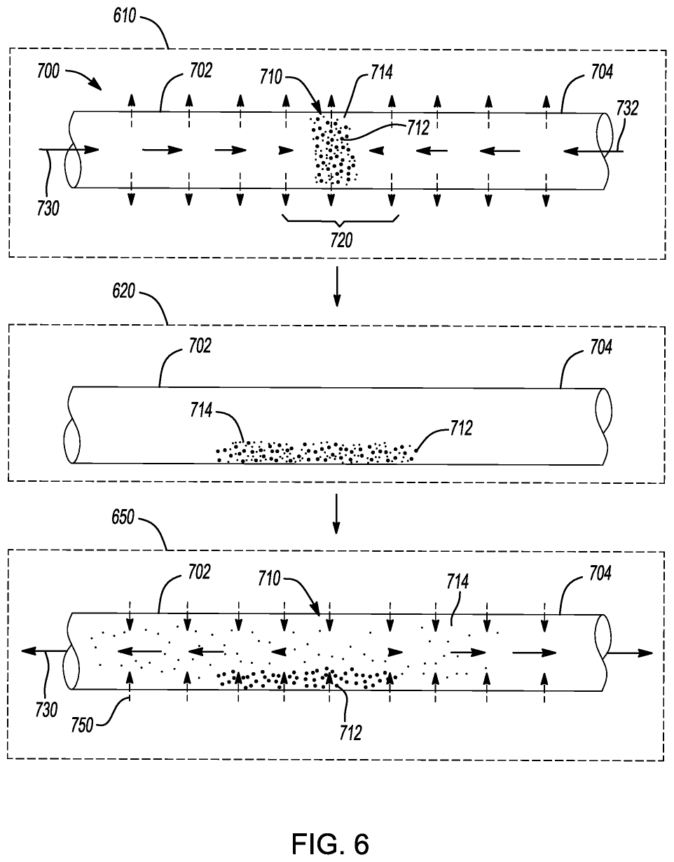

The present disclosure provides example methods for removing solutes (i.e., large molecular weight molecules) from an intracapillary space (or alternatively, or additionally, in the extracapillary space) of a bioreactor. For example, FIG. 5 is a flowchart illustrating a first example method 600 for removing solutes from an intracapillary space (or alternatively, or additionally, in the extracapillary space) of a bioreactor. The method 600 includes positioning 610 suspensions including cells and having an elevated concentration of solutes within a central region of a length of the bioreactor. In at least one example embodiment, positioning 610 the suspensions within the central regions of the length of the bioreactor may include using a counter flow confinement process or sequence that includes, for example, simultaneously pumping fluid into both ends of hollow fibers of the bioreactor.

For example, in reference to the example cell expansion system 500 illustrated in FIG. 4, positioning 610 the suspensions within the central regions of the length of the bioreactor may include setting the intracapillary inlet pump 554 to a first flow rate and setting intracapillary loop recirculation pump 512 to a second flow rate, where the second flow rate is equal to one half of the first flow rate. The intracapillary loop recirculation pump 512 may be set in a flow direction so as to pump a fluid into the intracapillary outlet port 501B of the cell growth chamber 501. Since the intracapillary loop recirculation pump 512 is removing only one half of the fluid arriving at connection 517, the remaining half of the flow will be forced to flow into the intracapillary inlet port 501A of the cell growth chamber 501. The fluid entering each end of the intracapillary space (e.g., via the intracapillary inlet port 501A and the intracapillary outlet port 501B) may be forced to cross the hollow fiber membrane (for example, via ultrafiltration), thus entering the extracapillary space and exiting the cell growth chamber 501 through the extracapillary inlet port 501C and/or the extracapillary outlet port 501D.

In at least one example embodiment, ultrafiltration may take place uniformly across the cumulative surface area of the hollow fibers. The pumping actions of intracapillary inlet pumps 554 and the intracapillary loop recirculation pump 512 may be coordinated to cooperate to increase fluid pressures within the intracapillary space to a apply a sufficient force to push water and low molecular weight solutes across the membrane. As the fluids escape across the hollow fiber membrane walls, the axial flow rate within each fiber automatically decreases from a maximum at the end of each fiber to zero at the mid-length (at mid-length because the flow into the intracapillary inlet port 501A and the intracapillary outlet port 501B are equal and in opposite directions) point of each fiber. This (uniform) decrease in axial flow rate may cause both cells and larger molecular weight solutes to accumulate and concentrate at a position equidistant between the intracapillary inlet port 501A and the intracapillary outlet port 501B.

With renewed reference to FIG. 5, once the suspensions are positioned 610 along the central region of the length of the bioreactor (i.e., region of the hollow fibers where the first and second flows meet), the method 600 may include positioning 620 the suspensions along fiber walls of the hollow fibers. In at least one example embodiment, positioning 620 the suspensions along the fiber walls of the hollow fibers may include placing the hollow fibers in a horizontal position and stopping fluid flow from the respective ends of the hollow fiber. In at least one example embodiment, the horizontal position may be along the central rotational axis 142, which is perpendicular to the force of gravity, as illustrated in FIG. 3. In at least one example embodiment, the horizontal positioning of the hollow fibers from a vertical positioning may occur as the fluid flow is stopped. In at least one example embodiment, the hollow fibers may be moved into the horizontal position using a rotatable fixture that is configured to hold the bioreactor. When the hollow fibers are orientated horizontally and the fluid flow is stopped, the suspensions having the elevated density of cells and solutes falls along the fiber walls of the respective hollow fibers. Once the suspensions fall, the higher concentration solutes, because of their molecular nature, will immediately begin to diffuse away from the cells, while the cells, because of their large size (compared to the solutes) and non-molecular nature, are not able to diffuse and will generally remain stationary.

Once the suspensions are positioned generally along the fiber walls of the hollow fiber, the method 600 may include opening 630 a first waste valve and closing 640 a second waste valve. The first waste valve may be an intracapillary waste valve (like the valve 590 illustrated in FIG. 4) and the second waste valve may be an extracapillary waste valve (like the valve 582 illustrated in FIG. 4). Once the first waste valve is opened 630 and the second waste valve is closed 640, the method 600 may include initiating 650 an inlet pump at a low flow rate that is sufficient to remove the solutes but retain the cells. For example, in at least one example embodiment, the flow rate may be greater than or equal to about 0.5 mL/min to less than or equal to about 5 mL/min. In at least one example embodiment, the inlet pump may be an intracapillary inlet pump, like the intracapillary inlet pump 554 illustrated in FIG. 4. For example, when the intracapillary inlet pump 554 provides fluid to the intracapillary loop 502, the intracapillary recirculation pump 512 may be stationary, and fluid may enter the intracapillary loop 502 via connection 517 and can then flows via valve 514 into the intracapillary inlet port 501A. Fluid carrying molecular solutes away from the cells may exit the cell growth chamber 501 via the intracapillary outlet port 501B. Because the waste valve 590 is open, fluid may move to the waste bag 586. In at least one example embodiment, the washing method 600 may be performed after or as the cell growth chamber 501 is placed at a 45-degree angle relative to the horizontal position, where the intracapillary outlet port 501B is positioned at a higher elevation than the intracapillary inlet port 501A.

When the inlet pump is an extracapillary inlet pump, such as pump 578 in FIG. 4, the inlet pump may generate an extracapillary flow that can enter the hollow fibers via reverse-ultrafiltration flow across the surface area of the hollow fibers. In this instance, the intracapillary inlet pump 554 and the intracapillary recirculation pump 512 may each have a zero flow rate. The waste valve 590 may be opened and the waste valve 582 may be closed. The ultrafiltration fluids crossing from the intracapillary space into the extracapillary space cannot exit the intracapillary inlet port 501A because the intracapillary inlet pump 554 and the intracapillary outlet pump 512 are stationary and blocking flow. The ultrafiltration fluids must then exit intracapillary outlet port 501B. The axial intracapillary flow rates within the hollow fibers may be in the direction from the intracapillary inlet port 501A towards the intracapillary outlet port 501B. Further, the axial flow rates may be increased from a minimum of zero immediately adjacent the intracapillary inlet port 501A to a maximum value immediately adjacent to intracapillary outlet port 501B. This is because the fiber lumens at the intracapillary outlet port 501B may accommodate the entire ultrafiltration volume flow rate, while, for example, fiber lumens between the intracapillary inlet port 501A and the intracapillary outlet port 501B need only accommodate one half of the ultrafiltration volume flow rate. As a result, velocities at the exit may be twice velocities between the EC ports. The flow rate generated by the extracapillary inlet pump may be about 100 mL/min and the membrane surface area may be about 2.1 square meters. In this example, a uniform radial flow velocity of about 0.005 cm/min through the walls of the hollow fibers may result. A nominally sized T-cell falls as the result of gravity at about 0.008 cm/min. In such instances, because radial flow velocities are too low to lift cells from the hollow fiber membrane walls, the flow velocities through the fiber walls may flow through the fallen bed of t-cells effectively washing solutes from between the T-cells.

In at least one example embodiment, the low flow rate may be optionally and periodically pulsed, so as to give the suspensions time to resettle near the fiber walls. In at least one example embodiment, the method 600 may include optionally titling the hollow fibers to migrate the cell bed (i.e., the suspension) towards the lower ends of the hollow fibers and biasing the fiber exit flow velocity to be greater at the elevated end of the hollow fibers to negate cell bed migration, thereby helping to retain the cells within the hollow fibers. This biasing can be accomplished by forcing all ultrafiltration flow to exit, for example, the intracapillary outlet port 501B by blocking flow from exiting the intracapillary inlet port 501A. In each instance, after at least a portion of the solutes are removed, as illustrated in FIG. 5, the method 600 may include resuming 660 normal cell expansion flows. In at least one example embodiment, resuming 760 normal cell expansion flow may include reorientating the hollowing fibers.

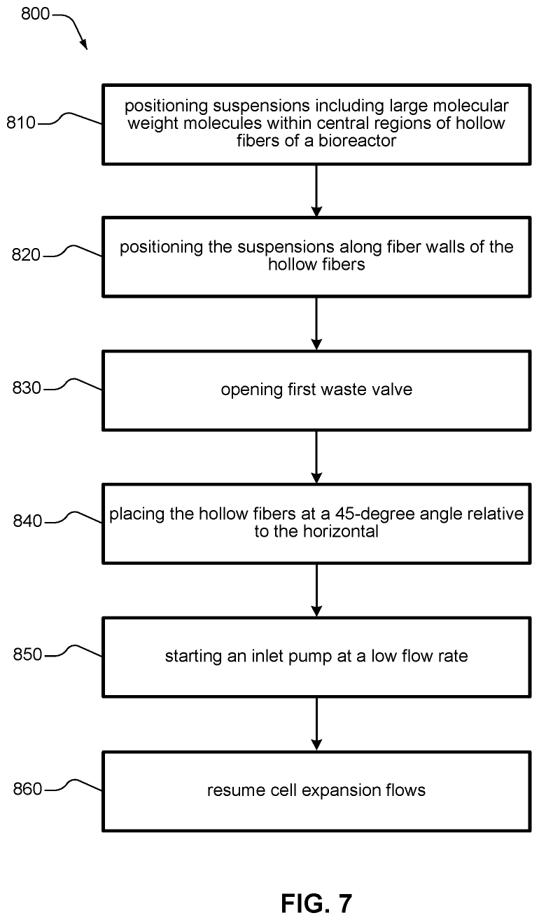

FIG. 6 is a simplified illustration of the method 600 as applied to a single hollow fiber 700. For example, as illustrated, a suspension 710 including cells 712 and solutes 714 may be positioned within a center region 720 of the hollow fiber 700 by pumping a first fluid flow 730 through a first end 702 of the hollow fiber 700 and pumping a second fluid flow 732 through a second end 704 of the hollow fiber 700. Although FIG. 6 shows the fiber in a horizontal orientation during the positioning, it should be appreciated that in at least one example embodiment, the fiber may be held instead in a vertical orientation. Once the suspension 710 is centered, the flow may be stopped (and if centered while in the vertical position, the fiber may be moved into a horizontal orientation) such that the suspension 702 falls against the fiber wall and a first waste valve may be opened (not shown) and a second waste valve may be closed (not shown), after which an inlet pump, like an extracapillary inlet pump, may be initiated to cause a low reverse ultrafiltration flow rate 750, such that the solutes 714 are pushed out of the first and second ends 702, 704 (for example, via exit flow 730) of the hollow fiber while the cells 712 generally remain along the fiber wall in the center region 720.

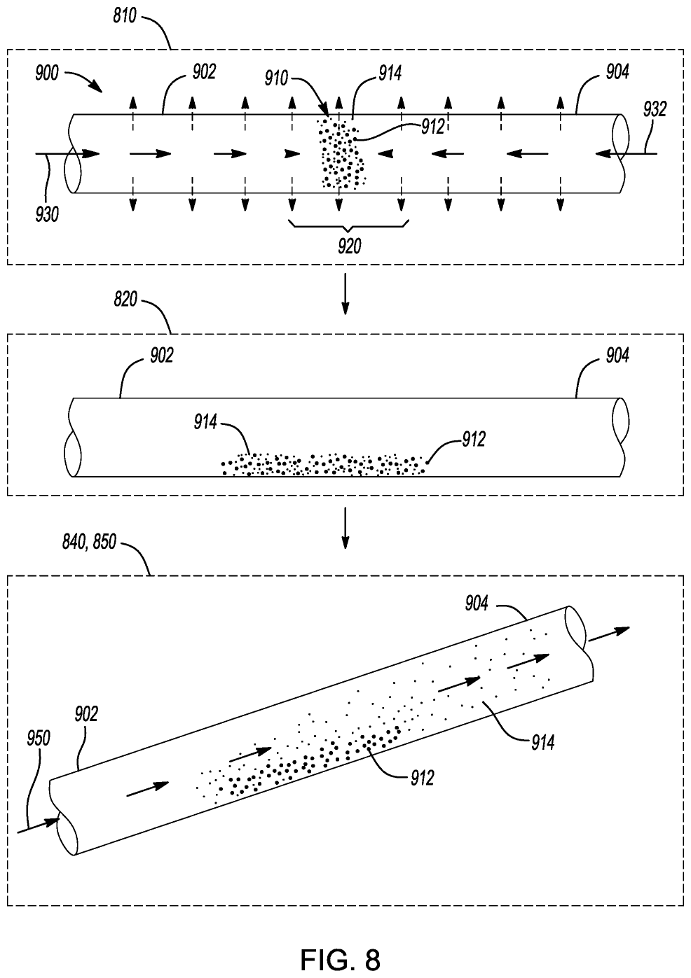

FIG. 7 is a flowchart illustrating a second example method 800 for removing solutes from an intracapillary space (or alternatively, or additionally, in the extracapillary space) of a bioreactor. Like method 600 illustrated in FIG. 5, method 800 may include first positioning 810 suspensions including cells and having an elevated concentration of solutes within a central region of a length of the bioreactor and then positioning 820 the suspensions along fiber walls of the hollow fibers. Also, like method 600, method 800 includes, once the suspensions are positioned along the fiber walls of the hollow fiber, opening 830 a first waste valve. Unlike the method 600, however, method 800 does not include closing a second waste valve, and includes, prior to initiating 850 an inlet pump, like an intracapillary inlet pump (e.g., intracapillary inlet pump 554 as illustrated in FIG. 4), at a low flow rate, placing 840 the hollow fibers at a 45-degee angle relative to the horizontal. The 45-degree angle may help to retain cells within the fiber by countering any drag forces imposed on the cells by the axial flow velocities imposed by the intracapillary inlet pump and/or an intracapillary recirculation pump (like the intracapillary recirculation pump 512 illustrated in FIG. 4). The forces imposed onto cells by the low axial flow rate may help to counter the tendency of the cells to slide to lower regions of the hollow fibers when in the angled position. In at least one example embodiment, the method 800 may optionally include moments of an increased flow rate to help accelerate the removal of the solute. In each instance, after at least a portion of the solutes are removed, the method 800 may include resuming 860 normal cell expansion flows. In at least one example embodiment, resuming 860 normal cell expansion flow may include reorientating the hollowing fibers.

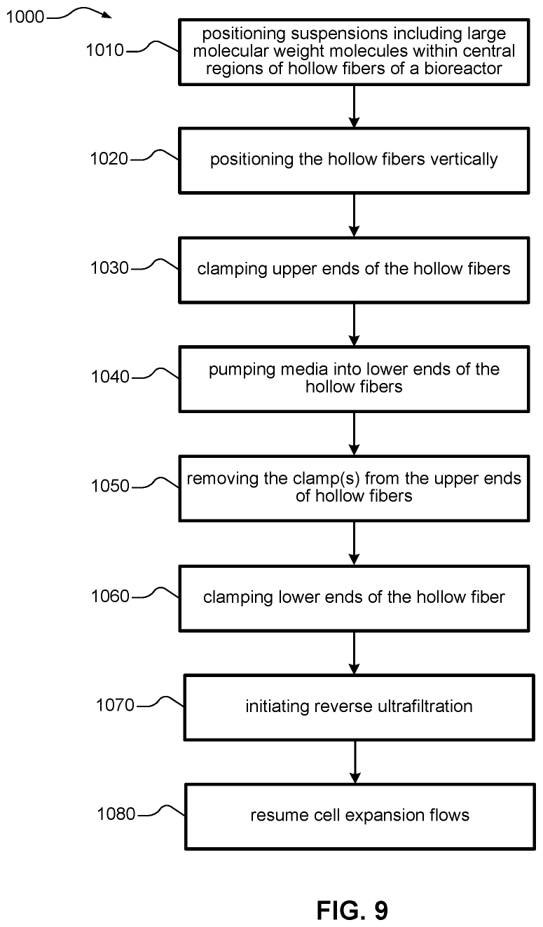

FIG. 8 is a simplified illustration of the method 800 as applied to a single hollow fiber 900. For example, as illustrated, a suspension 910 including cells 912 and solutes 914 may be positioned within a center region 920 of the hollow fiber 900 by pumping a first fluid flow 930 through a first end 902 of the hollow fiber 900 and pumping a second fluid flow 932 through a second end 904 of the hollow fiber 900. Although FIG. 8 shows the fiber in a horizontal orientation during the positioning, it should be appreciated that in at least one example embodiment, the fiber may be held instead in a vertical orientation. Once the suspension 910 is centered, the flow may be stopped (and if centered while in the vertical position, the fiber is moved into a horizontal orientation) such that the suspension 902 falls against the fiber wall and a waste valve may be opened (not shown), after which the hollow fiber may be placed at a 45-degee angle relative to the horizontal, such that the second end 904 is higher than the first end 902, and an inlet pump, like the intracapillary inlet pump, may be initiated at a low flow rate 950, such that the solutes 914 are pushed out of an upper end 904 of the hollow fiber, while the cells 912 generally remain along the fiber wall in the center region 920.

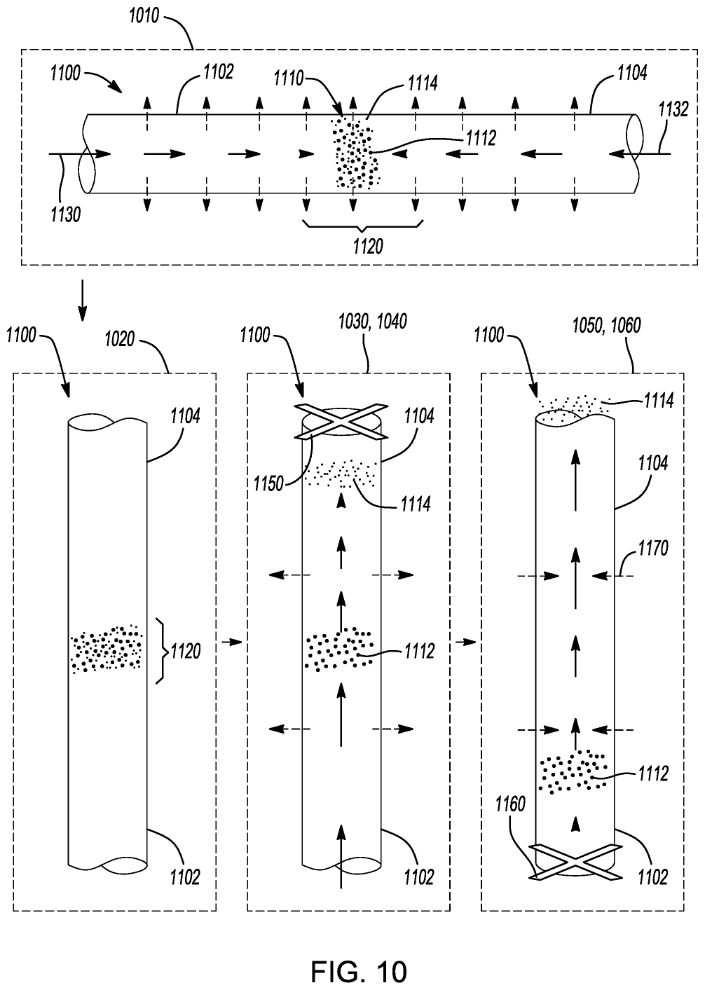

FIG. 9 is a flowchart illustrating a third example method 1000 for removing solutes from an intracapillary space (or alternatively, or additionally, in the extracapillary space) of a bioreactor. Like method 600 illustrated in FIG. 5 and/or the method 800 illustrated in FIG. 7, method 1000 may include first positioning 1010 suspensions including cells and having an elevated concentration of solutes within a central region of a length of the bioreactor. Unlike the method 600 illustrated in FIG. 5 and/or the method 800 illustrated in FIG. 6, the method 1000 does not include positioning 620, 820 the suspensions along fiber walls of the hollow fibers. Rather, the method 1000 includes positioning 1010 the suspensions in the central region of the length of the bioreactor and then positioning 1020 the hollow fibers vertically, which is generally perpendicular to the horizontal. Once the hollow fibers are positioned 1020 vertically, the method 1000 may include clamping 1030 upper ends of the hollow fibers and subsequently pumping 1040 media into lower ends of the hollow fibers. In at least one example embodiment, the upper end of the hollow fibers may be clamped 1030 by setting relevant pump rates to zero. Since the upper ends of the fibers are clamped, all fluid that enters the fibers must escape via ultrafiltration across the fiber walls. This forces a diminishing axial flow situation with maximum axial flow velocities at the entrance to fibers and zero axial flow velocities at the clamped ends of fibers. Because the axial flow velocity at the clamped upper end is zero, and the axial velocity continuously varies between fiber entrance and clamped end of fibers, cells will find a neutral position along the length of the fibers where cell sedimentation rate is equal to axial flow velocity, while solutes (with essentially zero sedimentation) will move towards and near the zero axial velocity region. In at least one example embodiment, the hollow fibers may be slighted tilted off of the vertical so to help the keep the cells in the neutral position. Once the cells and the solute are separated along the length of the fibers, the method 1000 may include removing 1050 the clamp(s) from the upper end and, subsequently or concurrently, clamping 1060 the lower ends of the hollow fiber, such that the cells, now experiencing zero axial flow rate, fall towards the clamped lower end and reverse ultrafiltration may be initiated 1070 to remove the solutes from the upper end of fibers. After at least a portion of the solutes are removed, the method 1000 may include resuming 1080 normal cell expansion flows. In at least one example embodiment, resuming 1070 normal cell expansion flow may include removing the clamp from the lower end and/or reorientating the hollowing fibers.

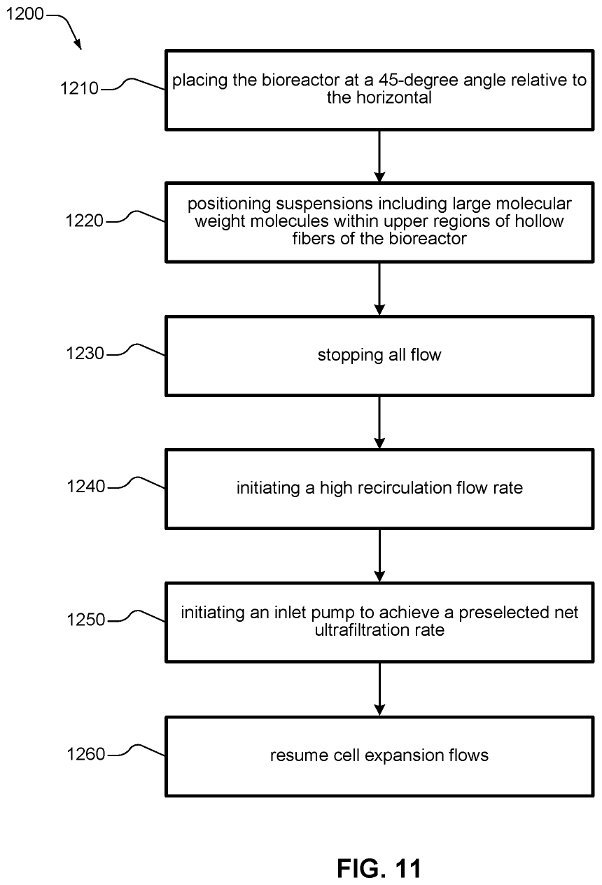

FIG. 10 is a simplified illustration of the method 1000 as applied to a single hollow fiber 1100. For example, as illustrated, a suspension 1110 including cells 1112 and solutes 1114 may be positioned within a center region 1120 of the hollow fiber 1100 by pumping a first fluid flow 1130 through a first end 1102 of the hollow fiber 1100 and pumping a second fluid flow 1132 through a second end 1104 of the hollow fiber 1100. Although FIG. 10 shows the fiber in a horizontal orientation during the positioning, it should be appreciated that in at least one example embodiment, the fiber may be held instead in a vertical orientation. Further, although FIG. 10 shows the suspension 1110 centered between the fiber ends 1102, 1104, it should be appreciated that in at least one example embodiment, the suspension might be positioned instead closer to one of the first and second ends 1102, 1104.

If the suspension 1110 was centered with the fiber in the horizontal orientation, the method 1000 may then include placing the hollow fiber 1100 in a generally vertical position where the second end 1104 is an upper end and the first end 1102 is a lower end. A first clamp 1150 may be applied to or near the upper end 1104 of the hollow fiber 1100 and a media pumped from the lower end 1102 to the upper end 1004. In at least one example embodiment, referring to FIG. 4, this may the accomplished by setting the intracapillary pump 512 to zero, setting the intracapillary inlet pump 554 to the desired ultrafiltration rate, closing the intracapillary waste valve 590, and opening the extracapillary waste valve 582. This flow maneuvering may result in the movement of the solutes 1114 from the center region 1120 towards and near the upper end 1004, while the cells 1112 remain in the center region and/or near the lower end 1002. Once the solutes 1114 are moved towards the upper end 1004, the first clamp 1150 may be removed and a second clamp 1160 may be applied to or near the lower end 1102 of the hollow fiber 1100, such that the cells 1112 fall towards the clamped lower end 1102. The second clamp 1160 may be the same as or different from the first clamp 1150. In at least one example embodiment, referring to FIG. 4, this may be accomplished by stopping the intracapillary inlet pump 554. Reverse ultrafiltration 1170 may then be initiated to remove solutes 1114 from the upper end of fibers 1104. In at least one example embodiment, referring to FIG. 4, reverse ultrafiltration 1170 may be initiated by stopping all pumps, closing the extracapillary waste valve 582, opening the intracapillary waste valve 590, and setting the extracapillary inlet pump 578 flow rate to the desired reverse ultrafiltration rate.

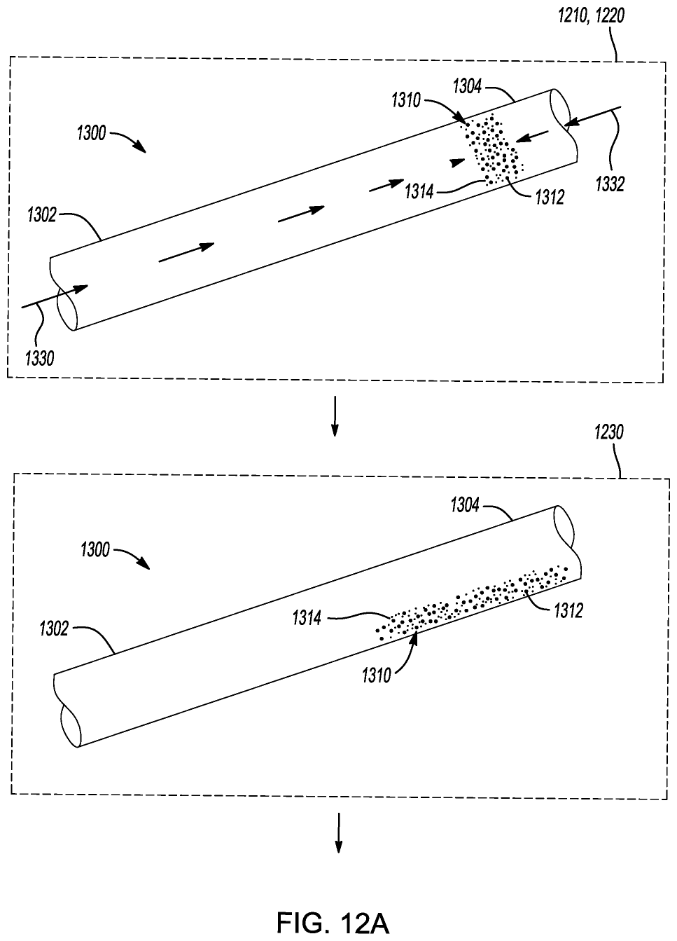

FIG. 11 is a flowchart illustrating a fourth example method 1200 for removing solutes from an intracapillary space (or alternatively, or additionally, in the extracapillary space) of a bioreactor. The method 1200 includes first positioning 1210 hollow fibers at a 45-degee angle relative to the horizontal and then positioning 1220 suspensions including cells and having an elevated concentration of solutes near upper ends of the hollow fibers. In at least one example embodiment, the suspensions may be positioned 1220 using a counter flow confinement process or sequence similar to that discussed in the context of method 600 and/or method 800 and/or method 1000. Once the suspension is positioned near the upper ends of the hollow fibers, the method 1200 may include: stopping 1230 all flow so as to cause the cells and solutes defining the suspension to start to fall down hollow fibers towards the lower ends; closing the extracapillary waste valve; closing the intracapillary waste valve; and initiating 1240 a high recirculation flow rate using the extracapillary recirculation pump (e.g., a high extracapillary recirculation flow rate). For example, with reference to FIG. 4, the high extracapillary flow rate may induce a pressure drop between the extracapillary inlet port 501C and the extracapillary outlet port 501D. The high pressure near the extracapillary inlet port 501C may induce reverse ultrafiltration (extracapillary to intracapillary) near the inlet. The lower pressure near the extracranially outlet port 501D may induce forward ultrafiltration (intracapillary to extracapillary) near the outlet. The axial flow within the hollow fibers may move the solutes toward the intracapillary outlet while the cells continue to fall down the fibers towards the intracapillary inlet. Once the high recirculation flow rate is established, the method 1200 may then include opening the intracapillary waste valve and initiating 1250 an inlet pump (e.g., an extracapillary inlet pump) to a preselected flow rate. In at least one example embodiment, the extracapillary inlet pump flow rate may be set to a flow rate of greater than or equal to about 1 ml/min to less than or equal to about 5 mL/min. This may result in a net reverse ultrafiltration (extracapillary to intracapillary) rate equal to the flow rate set on the extracapillary inlet pump. Because both the intracapillary pumps may be stopped, the only escape for the reverse ultrafiltration fluid is through the intracapillary outlet port 501B and intracapillary waste valve 590. Because of the (uniform) flow of fluid across the entire surface area of the hollow fibers, the axial flow rate may increase along the length of the hollow fibers and will wash out solutes while leaving behind the cells that have accumulated near the intracapillary inlet port 501A where axial flows are very low. The placement of the undesired solutes suspension near the upper ends of the hollow fibers (i.e., near the outlet of the hollow fiber) may mean that less ultrafiltrate volume and less time may be required to wash out the solute. After at least a portion of the solutes are removed, the method 1200 may include resuming 1260 normal cell expansion flows.

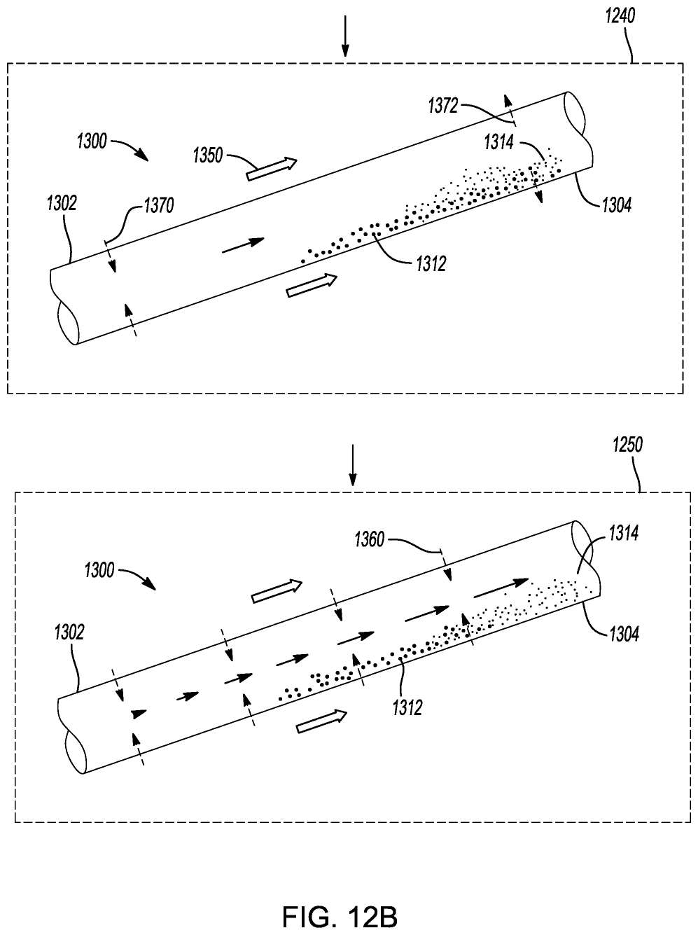

FIGS. 12A and 12B depict a simplified illustration of the method 1200 as applied to a single hollow fiber 1300. For example, as illustrated, a suspension 1310 including cells 1312 and solutes 1314 may be positioned near an upper end 1304 of a hollow fiber 1300 positioned at a 45-degee angle relative to the horizontal by pumping a first fluid flow 1330 through a first end 1302 of the hollow fiber 1300 and pumping a second fluid flow 1332 through a second end 1304 of the hollow fiber 1300. Once the suspension 1310 is positioned, the flow may be stopped such that the suspension 1310 starts to fall down hollow fibers towards the lower ends 1302 of the hollow fibers. A high recirculation flow rate 1350 is applied, for example, using the extracapillary recirculation pump 528, as illustrated in FIG. 5. The high recirculation flow may induce a pressure drop between the extracapillary inlet port 501C and extracapillary outlet port 501D so as to cause reverse ultrafiltration 1370 (extracapillary to intracapillary) at extracapillary inlet port 501C and forward ultrafiltration 1372 (i.e., intracapillary to extracapillary) at extracapillary outlet port 501D. Such a flow arrangement may move the solutes to the upper end of the fiber while the cells slide toward the lower end of the fiber. An extracapillary inlet pump 578 may be set to a preselected net ultrafiltration rate 1360 so as to wash out solutes while leaving behind the cells.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A method for removing solutes while retaining suspension cells within a bioreactor, the method comprising:
positioning a suspension including cells and solutes within a predetermined region of the bioreactor; and
causing a flow rate across the suspension in the bioreactor, the flow rate being selected to cause the solutes to move out of the bioreactor while the cells are maintained in the bioreactor.

2. The method of claim 1, wherein the positioning of the suspension includes:
applying a first flow to first ends of the bioreactor; and
applying a second flow to second ends of the bioreactor,
the first rate and the second flow together modifying a first axial flow from the first ends and a second axial flow from the second ends such that the suspension is positioned within the predetermined region of the bioreactor.

3. The method of claim 2, wherein the first flow has a first flow rate and the second flow has a second flow rate that is different from the first flow rate.

4. The method of claim 3, wherein the second flow rate is less than the first flow rate.

5. The method of claim 4, wherein the second flow rate is less than one half of the first flow rate.

6. The method of claim 2, wherein the positioning of the suspension further includes:
positioning the bioreactor in a horizonal or a near horizontal orientation such that the suspension falls onto or near a wall defining the bioreactor.

7. The method of claim 6, wherein after the positioning of the suspension and before the causing of the flow across the suspension, the method further include:
stopping all flows through the bioreactor.

8. The method of claim 7, wherein after the stopping of all flows through the bioreactor and before the causing of the flow across the suspension, the method further includes:
positioning flow valves to enable flows to exit the bioreactor.

9. The method of claim 7, wherein before the causing of the flow across the suspension, the method may further include:
placing the bioreactor at a 45-degee or a near 45-degree angle relative to the horizontal.

10. The method of claim 1, wherein the predetermined region of the bioreactor is a position central between first ends and opposing second ends of the bioreactor.

11. The method of claim 10, wherein the predetermined region is perpendicular to a major axis of the bioreactor.

12. A method for removing solutes while retaining suspension cells within a bioreactor, the method comprising:
positioning a suspension including cells and solutes within a predetermined region of the bioreactor;
positioning the bioreactor to create an upper end and a lower end of the bioreactor;
preventing flow from the upper end of the bioreactor;
flowing media into the lower end of the bioreactor;
enabling flow from the upper end of the bioreactor;
preventing flow from the lower end of the bioreactor; and
flowing fluid across the bioreactor to cause the solutes to move out of the bioreactor and the cells to remain in the bioreactor.

13. The method of claim 12, wherein the predetermined region of the bioreactor is a position central between first ends and opposing second ends of the bioreactor.

14. The method of claim 12, wherein the predetermined region is perpendicular to a major axis of the bioreactor.

15. A method for removing solutes while retaining suspension cells within a bioreactor, the method comprising:
orientating a bioreactor at an angle relative to a horizontal creating upper and lower ends of the bioreactor;
positioning a suspension including cells and solutes in a predetermined position within the bioreactor;
initiating a recirculation flow rate; and
initiating a preselected net ultrafiltration rate.

16. The method of claim 15, wherein the positioning of the suspension includes:
applying a first flow to lower ends of the bioreactor; and
applying a second flow to the upper end of the bioreactor,
the first rate and the second flow together modifying a first axial flow from the first ends and a second axial flow from the second ends such that the suspension is positioned within the predetermined region of the bioreactor.

17. The method of claim 16, wherein the first flow has a first flow rate and the second flow has a second flow rate that is different from the first flow rate.

18. The method of claim 17, wherein the second flow rate is less than the first flow rate.

19. The method of claim 18, wherein the second flow rate is less than one half of the first flow rate.

20. The method of claim 15, wherein after the positioning of the suspension in the predetermined position within the bioreactor and before the initiating of the recirculation flow rate, the method further includes:
stopping all flow rates.
